(19) United States
(12) Patent Application Publication
(10) Pub. No.: US 2021/0386583 A1
(43) Pub. Date: Dec. 16, 2021

(54) VITRECTOMY NEEDLE, A VITRECTOME, A VITRECTOMY DEVICE AND A METHOD OF MANUFACTURING A VITRECTOMY NEEDLE

(71) Applicant: A.R.C. Laser GmbH, Nürnberg or Nuremberg? (DE)

(72) Inventor: Reinhardt Thyzel, Eckental (DE)

(21) Appl. No.: 17/345,952

(22) Filed: Jun. 11, 2021

(30) Foreign Application Priority Data
Jun. 16, 2020 (DE) 10 2020 115 885.8

(51) Int. Cl. A61F 9/008; A61F 9/007

(52) U.S. Cl. CPC A61F 9/008; A61F 2009/00874; A61F 9/00763

(57) ABSTRACT

A vitrectomy needle comprises a hollow needle with a distal end for insertion into the vitreous body of an eye for performing a vitrectomy. The vitrectomy needle further comprises a laser light guide guided in the hollow needle to the distal end and has a light-emitting surface oriented towards the distal end. A cavity is formed between the light-emitting surface and the axially inward-facing surface of the distal end. In the area of the cavity, the wall of the hollow needle has an aperture extending radially with respect to the middle axis (M) of the hollow needle. The vitrectomy needle, has a straight section at the proximal end, the middle axis (M) defining a central axis (Z) in the region of the straight section. The middle axis (M) of the hollow needle may be spaced apart from the central axis (Z) by a predetermined minimum radial distance (R).

### CROSS-REFERENCE TO RELATED APPLICATIONS

The present invention claims the benefit of priority to German Patent Application No. 10 2020 115 885.8, filed on Jun. 16, 2020, entitled “Vitrectomy Needle, A Vitrectome, A Vitrectomy Device and A Method of Manufacturing a Vitrectomy Needle,” the entire content which is incorporated herein by reference.

### BACKGROUND OF THE INVENTION

### 1. Field of the Invention

The invention relates to a vitrectomy needle, a vitrectome, a vitrectomy device and a method of manufacturing a vitrectomy needle.

During vitrectomy, the vitreous body (corpus vitreum) of the eye is removed successively or in small segments (ablation) using an instrument called a vitrectome or vitrector.

Vitrectomy is in particular used in the treatment of diseases or detachments of the retina or of bleeding opacity. Three small incisions are made in the sclera in an area called pars plana as access points for the vitrectomy instrument, for a light source and for an infusion and suction tool. Small access sleeves, so-called trocars, are inserted into these accesses through which the instruments are introduced into the eyeball.

During the operation, the pressure in the eye socket compartment is maintained by infusing a solution consisting mainly of water. The cavity created after the vitreous body has been removed is usually filled with a substitute material, for example silicone oil or also gas or perfluorocarbons, which exerts a corresponding contact pressure on the retina, which can then be welded on again by laser coagulation, for example.

The vitrectomes used in practice work mechanically and include small blades or cutting edges or forceps or hooks which are moved backwards and forwards within a straight tube by means of a carrier, the segments of vitreous body thus ablated or cut off then being aspirated by means of the suction instrument or by the vitrectome itself.

WO 2015/135544 A1 discloses a vitrectome comprising mechanical moving cutting elements for separating vitreous material in vivo.

Known vitrectomes are usually only straight or rectilinear because of the cutting mechanism. A curved vitrectome with a mechanical cutting mechanism is known from US 2017/0071788 A1.

During vitrectomy, the vitrectome is passed through a trocar and moved piece by piece in the vitreous body until the entire vitreous body or a part to be removed is removed. In the case of straight vitrectomes, a fairly wide solid angle or swivel range of the vitrectome must be used to reach all areas of the vitreous body. This leads to considerable forces in the area of the trocar and the surrounding tissue and can result in the entrance incision through which the trocar is passed lacerating further and requiring suturing. Mechanical vitrectomies have the additional disadvantage that the mechanical cutting movements can lead to further mechanical stress in the eye and in the area of the trocar.

In DE 20 2018 105 448 U1, a vitrectome designed as a laser instrument is proposed. It works with photoemulsification, similar to that already used for phacoemulsification of cataracts. This laser instrument is straight and, like the straight vitrectomes, is moved in a trocar by means of a pivoting movement. In the laser instrument for vitrectomy known from DE 20 2018 105 448 U1, the exit opening of the hollow needle to be introduced into the eye is located in a lateral region of the end region of the hollow needle. The distance of this opening from the central axis of the hollow needle corresponds to half the diameter of the hollow needle of the laser instrument in a range of 0.175 to 0.375 mm.

With phacoemulsification, there is no problem at all in reaching all areas of the comparatively very small lens with the straight laser instrument. Here there is also no risk of injury of or of lacerating the entrance incision in the cornea during the operation because the pivoting movement of the instrument is very small due to the small size of the lens. With vitrectomy, however, due to the much greater pivoting movement, considerably larger deflections occur through the vitrectome in the area of the incision and therefore the lacerations already described often occur, which have to be sutured.

### BRIEF SUMMARY OF THE INVENTION

It is therefore an object of the invention to provide a vitrectomy needle, a vitrectome, a vitrectomy device and a method of manufacturing a vitrectomy needle with which the disadvantages of the prior art can be eliminated. In particular, a vitrectomy needle, a vitrectome and a vitrectomy device are to be provided which enable a comparatively gentle removal of the vitreous body for the eye to be treated. Furthermore, in particular a vitrectomy needle, a vitrectome and a vitrectomy device shall be provided which enable a particularly efficient removal of the vitreous body and offer a simplified, in particular ergonomically improved handling for the applying physician or operator when performing a vitrectomy. Furthermore, a method of manufacturing a corresponding vitrectomy needle shall be provided.

This object is solved by the independent patent claims. Embodiments result from the dependent patent claims as well as from the following description.

According to an embodiment, a vitrectomy needle is provided. The vitrectomy needle comprises a hollow needle, for example in the form of a cannula or a thin, thin-walled tubular hollow body.

The hollow needle comprises a distal end for insertion into the vitreous body of an eye for performing a vitrectomy. In particular, the vitrectomy needle is designed such that it can be inserted with the distal end through a trocar into the eye and into the vitreous body to perform a vitrectomy.

Furthermore, the vitrectomy needle comprises a laser light guide guided in the hollow needle to the distal end. The laser light guide is guided in the hollow needle and fixed therein in a suitable manner. In particular, the laser light guide may be secured by one or more adhesive attachments. The laser light guide may, for example, have an outer diameter of 250 μm to 300 μm, in particular of about 270 μm. The diameter of the laser light guide may be selected depending on the inner diameter and/or outer diameter of the hollow needle, preferably the larger the inner or outer diameter of the hollow needle, the larger the diameter of the laser light guide. For example, with a 23 G hollow needle (0.6 mm outer diameter), the laser light guide can have an outer diameter of about 270 μm.

The laser light guide has a light-emitting surface oriented towards the distal end, wherein a cavity is formed in the hollow needle in the region of the light-emitting surface and the axially inward-facing surface of the distal end, in particular between the light-emitting surface and the inward-facing surface of the distal end. For example, the laser light guide may be formed, arranged and positioned such that the light-emitting surface of the laser light guide is spaced from the axial inner wall at the closed end of the distal end by 0.9 mm to 1.2 mm. Preferably, the distance between the light-emitting surface depends on the outer diameter of the hollow needle, and is the smaller the smaller the outer diameter.

In the area of the cavity, the wall of the hollow needle has an aperture, in particular an outlet opening, which extends radially with respect to the middle axis of the hollow needle.

The hollow needle has a straight section at the proximal end, i.e. the end remote from the distal end. The proximal end of the hollow needle may, for example, be adapted to be attached to or with a handpiece or handle. The straight section may, for example, be adapted to be located or positioned in a trocar when performing a vitrectomy on the eye.

The middle axis of the hollow needle defines a central axis in the area of the straight section of the proximal end.

With the vitrectomy needle, it is provided that the middle axis of the hollow needle is spaced apart from the central axis by a predetermined minimum radial distance in the area of the aperture or cavity. The predetermined minimum distance can be achieved, as described in more detail below, for example by the hollow needle being curved or bent. It is also possible that the vitrectomy needle, starting from the straight section, is, at least in sections, oblique, in particular at an acute angle, to the central axis.

With the predetermined minimum radial distance, the forces that act in the area of the entry incision of the vitrectomy needle into the eye when performing a vitrectomy, and thus the risk of lacerating the entry incision or an associated wound may be reduced. In particular, the fact that the aperture, i.e. the working area defined by the aperture, of the vitrectomy needle is offset radially further outwards in relation to the central axis means that a significantly larger solid angle range is possible simply by rotating the vitrectomy needle about the central axis. Consequently, swivelling movements, which generate forces acting on the entrance incision, are only necessary to a small extent, or may possibly even avoided entirely.

Indeed, the insertion of the vitrectomy needle or a corresponding laser instrument into the eye, for example through a trocar, requires greater attention, because a somewhat more complicated movement is required compared to a continuous straight instrument. However, the advantages achieved after inserting the vitrectomy needle into the eye, such as the significantly simplified handling and the significantly reduced force input into the entrance incision when performing the vitrectomy, are clearly predominant.

In order to simplify and facilitate the insertion of the vitrectomy needle, the vitrectomy needle can be specially shaped, as will be explained in more detail below. For example, the vitrectomy needle may be curved so that a continuous and, in particular, edge-free shape of the vitrectomy needle enables a correspondingly smooth and continuous insertion movement. Such movements are particularly advantageous in terms of reducing the forces that may act on the entry incision during insertion.

When performing a vitrectomy, the vitrectomy needle is inserted into the eye through an entry incision in the area of the eye, for example using a trocar, so that the distal having the aperture is positioned within the vitreous body of the eye. The laser light guide is then exposed to laser light, preferably laser pulses, through a laser light source coupled to it. For example, a pulse laser, in particular a Nd:YAG pulse laser, may be used as the laser light source. The laser pulses may be generated, for example, with a pulse frequency of 40 Hz to 60 Hz and a pulse energy in the range of 0.5 to 3 mJ, in particular in the range of 1 to 2 mJ.

The laser pulses emitted from the light-emitting surface of the laser light guide inside the vitrectomy needle generate, in the area of the cavity and the aperture, shock waves which exit through the aperture and correspondingly shatter or fragment the vitreous body material. Thus, the glass body material is photolytically degraded or fragmented. The degraded or fragmented vitreous material can be aspirated by means of a suitable aspiration device, which can be integrated in a handpiece or a handle or designed as a separate instrument. To maintain the intraocular pressure during vitrectomy, the removed vitreous body material is usually replaced with gas or a liquid, in particular silicone oil (irrigation).

According to embodiments, the hollow needle may have a curvature, in particular a curved course, for example with at least one bend, with a, preferably constant, radius of curvature, starting from the straight section at the proximal end towards the distal end. As already mentioned, despite the curvature or curved course, vitrectomy needles shaped in this way may be inserted into the eye comparatively easily, for example in a substantially continuous sequence of movements, through the entry incision, in particular a trocar. Unnecessary forces on the entry incision can be at least largely avoided.

In embodiments, the curvature or curved course extends from the straight section substantially to the distal end. In embodiments, the curvature may be a continuous curvature from the straight section to the distal end.

By this, it is in particular possible to obtain a continuous motion execution when inserting the vitrectomy needle. Furthermore, with a suitable curvature, a radius of action that is advantageous for the removal of the vitreous body can be achieved just by rotating the vitrectomy needle around the central axis, so that the number of required tilts or swivels of the vitrectomy needle may be significantly reduced. The advantages mentioned apply accordingly to the geometries and shapes of the vitrectomy needle described above and below.

According to embodiments, the hollow needle may have at least one further straight section starting from the curvature towards the distal end. In this case, there is no continuous curvature.

For example, the straight section starting from the proximal end can be followed by a curvature, which is followed by another straight section towards the distal end. Despite the deviation from a completely straight shape, also such geometries and shapes enable a comparatively simple insertion of the vitrectomy needle and, at the same time, an advantageous radius of action of the working area, i.e. the aperture of the hollow needle.

According to embodiments, the hollow needle has an outer diameter ranging from 0.55 mm to 0.65 mm, preferably 0.6 mm. According to further embodiments, the hollow needle may have an inner diameter ranging from 0.35 mm to 0.55 mm, in particular from 0.35 mm to 0.45 mm, preferably about 0.4 mm. In further embodiments, the hollow needle may have a length measured along the middle axis ranging from 18 mm to 30 mm, in particular between 20 mm and 27 mm, preferably about 25 mm. In particular, such vitrectomy needles allow minimally invasive procedures in connection with the removal of the vitreous body or parts thereof, for example in the case of entry incisions in the region of the pars plana.

According to embodiments, the minimum radial distance is in the range of at least 5 mm to 30 mm. In particular, it is within the scope of the invention that it is possible to manufacture vitrectomy needles according to the described geometries and shapes having the mentioned minimum radial distances. Thus, it is possible to manufacture vitrectomy needles with different action radii with respect to a rotation around the central axis for different applications and/or different eye sizes.

According to embodiments, the aperture may be formed on a side of the hollow needle facing the central axis, or on a side of the hollow needle facing away from the central axis. Through these two variants, the specificity of application of the vitrectomy needle may, in particular, be adapted in an advantageous manner. For example, vitrectomy needles with apertures positioned differently may be provided, respectively particularly suitable for removing particular areas of the vitreous body. It is conceivable, for example, that vitrectomy needles with apertures facing away from the central axis, for example on a concavely curved section of the hollow needle, are used for removing areas located centrally in the vitreous body, and that vitrectomy needles with apertures facing the central axis, for example on a convexly curved section of the hollow needle, are used for removing areas located at the edge of the vitreous body. However, both embodiments are equally suitable for removing the entire vitreous body, although the type and extent of the pivoting movements required may differ.

According to an embodiment, a vitrectome, i.e. a surgical instrument for performing a vitrectomy, is provided, wherein the vitrectome comprises a handpiece and, connected to the handpiece, a vitrectomy needle according to one of the embodiments according to the invention described herein. Advantages and beneficial effects of the vitrectomy needle described in connection with the invention apply accordingly to the vitrectome.

According to embodiments, the vitrectomy needle may be detachably attached to the handpiece, in particular by means of a screw connector formed at the proximal end of the vitrectomy needle, is the screw connector adapted to be screwed to the handpiece, for example. In embodiments, the vitrectomy needle may be firmly connected to the handpiece, for example by means of an adhesive connection.

According to embodiments, a vitrectomy device is provided comprising a laser light source, a control unit for controlling the laser light source and a connection interface for connecting a vitrectome according to one of the embodiments of the invention described herein. A vitrectome may be connected to the connection interface. The control unit is adapted to control the laser light source to perform a vitrectomy when the vitrectome is connected. For example, the control unit may control the laser light source, for example according to an activation via a user interface, in such a way that it emits laser light pulses that are suitable for performing a vitrectomy, and may then be applied via the vitrectomy needle.

The vitrectomy device may include other components necessary for performing a vitrectomy, such as an aspiration device for aspirating shattered vitreous material, and an irrigation device for introducing irrigation fluid, etc.

According to an embodiment, there is further provided a method of manufacturing a vitrectomy needle according to any of the embodiments proposed herein in accordance with the invention. The method comprises the following steps: providing an elongated solid, in particular cylindrical, blank; creating a hollow body from the blank by drilling a central blind hole extending in the longitudinal direction of the blank by means of a drilling tool; creating, with respect to the longitudinal direction, a radial aperture at the closed end of the blind hole by means of a milling tool in such a way that the aperture opens radially into the blind hole; heating the hollow body to a forming temperature, generally and in particular in the range between 60° C. and 140° C., preferably in the range between 100° C. and 130° C., more preferably at about 120° C.; and forming of the hollow body to the final geometry of the vitrectomy needle by means of a forming tool. Preferably, the blank used for manufacturing the vitrectomy needle has an outer diameter of 0.2 mm to 1 mm.

The radial aperture is advantageously created before the forming, i.e. before the forming step.

The use of a blind hole, i.e. an axial hole that does not extend through the entire blank, in particular has the advantage that the hollow body is already closed at the distal, axial end, and a separate process step for closing the distal end is not necessary in this respect.

The forming tool or tools used for forming the vitrectomy needle, such as dies, may for example be configured for forming, on the one hand, the outer contour and, on the other hand, the inner contour.

The hollow body may be heated resistively electrically (ohmic) and/or inductively, for example.

### BRIEF DESCRIPTION OF THE DRAWINGS

Exemplary embodiments of the invention are described below with reference to the accompanying figures, in which:

FIG. 1 is a vitrectomy needle according to a first embodiment of the invention;

FIG. 2 a vitrectomy needle according to a second embodiment of the invention;

FIG. 3 is an enlarged view of a vitrectomy needle with exemplary dimensions;

FIGS. 4 and 5 two further embodiment variants of a vitrectomy needle;

FIG. 6 a schematic view of a vitrectomy device with vitrectome;

FIG. 7 a use of the vitrectomy needle, in a schematic view and not to scale;

FIG. 8 another embodiment of a vitrectome;

FIG. 9 an enlarged portion of the distal end of the vitrectomy needle of the vitrectome of FIG. 8; and

FIG. 10 process steps of a method of manufacturing the vitrectomy needle.

Unless otherwise explicitly described, identical or functionally identical elements are designated with the same reference signs in the figures. Furthermore, the figures are not necessarily true to scale.

### DETAILED DESCRIPTION OF THE PREFERRED EMBODIMENTS

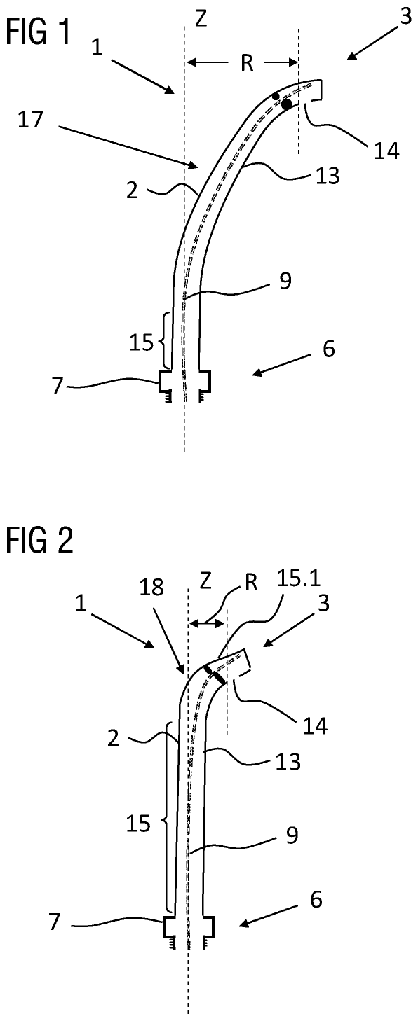

FIG. 1 shows a vitrectomy needle 1 according to a first embodiment of the invention. The vitrectomy needle 1 comprises a hollow needle 2 with a distal end 3 for insertion into the vitreous body 4 of an eye 5 for performing a vitrectomy, which is in particular also shown in FIG. 7.

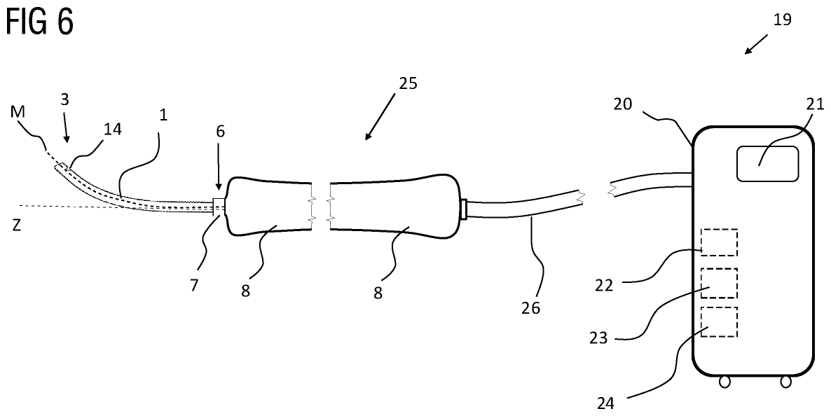

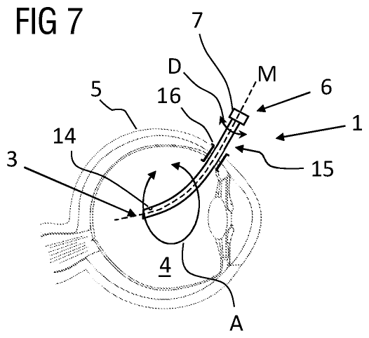

The vitrectomy needle 1 shown in the figures comprises, by way of example, at the proximal end 6 of the vitrectomy needle 1 facing away from the distal end 3, a screw connector 7 for detachable attachment to a handle part 8 shown in FIG. 6. Other possibilities for attachment, such as adhesive connections, may also be considered as alternatives or supplements for attaching the vitrectomy needle to a handle part and/or a holder. In particular, the vitrectomy needle may be glued on or glued to the handle.

The vitrectomy needle 1 further comprises a laser light guide 9 guided in the hollow needle 2 to the distal end 3 and fastened there by means of suitable fastening elements, with a light-emitting surface 10 being oriented towards the distal end 3.

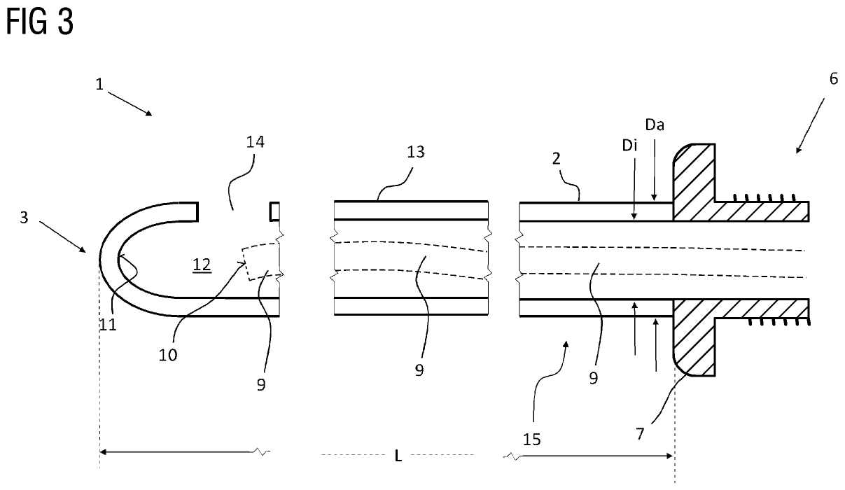

As can be seen in particular from FIG. 3, a cavity 12 is formed between the light-emitting surface 10 and the axially inward-facing surface 11 of the distal end 3. Furthermore, in the region of the cavity 12, the wall 13 of the hollow needle 2 has an aperture 14 extending radially with respect to the middle axis M of the hollow needle 2, which is only shown in FIG. 6 and FIG. 7 for reasons of clarity. The middle axis M of the hollow needle 2 or of the vitrectomy needle 1 is to be understood as the axis that runs centrally through the hollow needle 2 or the vitrectomy needle 1 following the shape of the hollow needle 2 or vitrectomy needle in its longitudinal direction.

At the proximal end 6, the hollow needle 2, in particular the vitrectomy needle 1, has a straight section 15. The straight section may, for example, be a connector, such as a screw connector 7, for connection to a handle part 8 (FIG. 6). However, the straight section 15 may also be a section of the hollow needle 2, as shown in the figures.

As can be seen from the figures, the straight section 15 may have different lengths in different embodiments of the hollow needle 2. In the embodiments shown, the straight section 15 of the hollow needle 2 has a minimum length which corresponds at least to the axial length of a trocar 16, which axial length may for example be in the range from 20 nm to 30 nm, with the trocar 16 through which the hollow needle 2 is inserted into the eye 5 during a vitrectomy being schematically shown in FIG. 7. With the straight section 15, the hollow needle 2 may be rotated about the central axis Z of the vitrectomy needle 1 or the hollow needle 2 after having passed through the trocar 16, without tilting moments being exerted on the trocar 16.

In the context of the invention, the central axis Z is to be understood as the axis which is defined by, or runs parallel to, the middle axis M in the straight section 15 of the proximal end 6 of the hollow needle 2, or vitrectomy needle 1. In particular, the central axis Z is to be understood as the axis that is defined by the straight line running collinearly to the middle axis M in the straight section 15 at the proximal end 6.

In the vitrectomy needles 1 of the embodiments according to FIGS. 1 to 7, the middle axis M of the hollow needle 2 or of the vitrectomy needle 1 is spaced apart from the central axis Z by a predetermined minimum radial distance R in the region of the aperture 14. The minimum radial distance R is shown in the figures with respect to the edge of the aperture 14 facing the proximal end. It is also possible that the centre of the opening surface of the aperture 14 is considered for the minimum radial distance R. The term “predetermined minimum radial distance” shall in particular be understood to mean that the minimum distance is not a random deviation of the central axis Z from the middle axis M, but a specific and predetermined distance set during the manufacture of the vitrectomy needle 1 or the hollow needle 2.

As mentioned and discussed above, the described deviation of the middle axis M from the central axis Z offers advantages with regard to the handling of the vitrectomy needle 1 as well as with regard to the forces acting on the entry incision of the eye 5.

The vitrectomy needle 1 shown in FIG. 1 has a continuous curvature 17 starting from the straight section 15 towards the distal end 3, the radius of curvature varying along the middle axis M.

Compared to the vitrectomy needle 1 of FIG. 1, the straight section 15 of the hollow needle 2 of the embodiment according to FIG. 2 is elongated. The straight section 15 of the hollow needle 2 of FIG. 2 is followed by a bend 18, which in turn is followed by a further, albeit short, straight section 15.1.

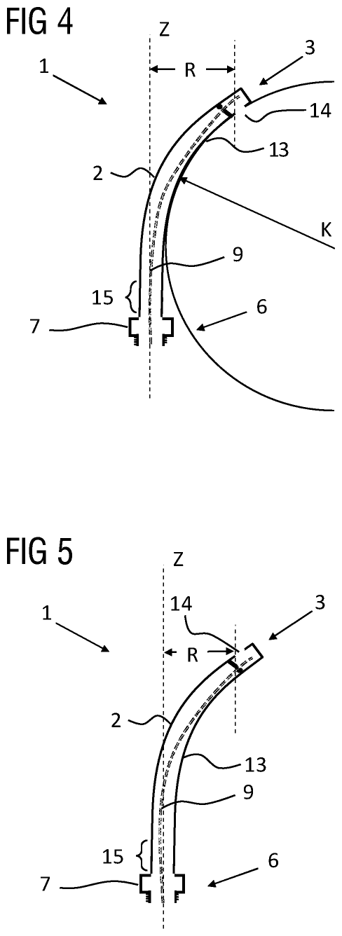

In the embodiment according to FIGS. 4 to 7, the hollow needle 2 has a curvature with continuous curvature starting from the straight section 15 towards the distal end 3, wherein, in addition, a substantially constant radius of curvature K is implemented. For clarity, the radius of curvature K is only shown in FIG. 4.

The embodiments according to FIG. 4 and FIG. 5 differ only in the position of the aperture 14 relative to the central axis Z. Whereas in FIG. 4 the aperture 14 is provided on a side of the hollow needle 2 facing away from the central axis Z, in FIG. 5, the aperture 14 is provided on the side of the hollow needle 2 facing towards the central axis Z.

Exemplary dimensions of the vitrectomy needle 1 or the hollow needle 2 are shown in FIG. 3. According to FIG. 3, the hollow needle 2 may have an outer diameter Da of 0.4 mm. An inner diameter Di of the hollow needle 2 may be 0.25 mm. Measured along the middle axis M, i.e. following the shape of the hollow needle 2, the hollow needle 2 may have a length L of, for example, 25 mm.

A wall thickness of the vitrectomy needle may be about 0.1 mm, for example.

Based on medical terminology, the size of the vitrectomy needle may be in the range from 20 G to 27 G (G: gauge). These sizes correspond to outer diameters in the range between 0.4 mm (at 27 G) and 0.9 mm (at 20 G).

The minimum radial distance shown with reference to FIGS. 1, 2, 3 and 4 may be in the range of at least 5 mm to 30 mm.

FIG. 6 shows a vitrectomy device 19 comprising a mobile central unit 20 with a control panel 21, a laser light source 22, an aspiration and irrigation device 23 and a control unit 24, which is set up to control the laser light source 22, the aspiration and irrigation device 23 and possibly other components and units for performing a vitrectomy when the vitrectome 25 is connected. For this purpose, the control unit 24 may comprise a memory with computer-readable instructions stored thereon which, when executed by a processor, may control the corresponding units for performing a vitrectomy, in particular in accordance with a user input via the control panel 21 and/or via the handle part 8 of the vitrectome 25.

The vitrectome 25 comprises the or a vitrectomy needle 1, and the handle part 8 connected to the vitrectomy needle 1. For example, the vitrectomy needle 1 may be firmly connected to the handle part by an adhesive connection. Other types of connection are also or alternatively possible, as described further above. Through and connected to the handle part 8 is a control and/or signal line 26, hereinafter referred to as line for short, which in turn is connected to the central unit 20. The line 26 is at least designed to transmit laser light, in particular laser pulses, from the laser light source 22 to the laser light guide 9 of the vitrectomy needle 1. For example, a laser light guide (not shown) may be present in the line 26 itself, which laser light guide is coupled to the laser light guide 9 when the vitrectomy needle 1 is connected.

During operation of the vitrectomy device 19, laser pulses are generated by the laser light source 22, for example, and coupled into the laser light guide 9 of the vitrectomy needle 1 through the line 26. Laser pulses emitted at the light-emitting surface 10 cause photolytic fragmentation of the vitreous material, for example by laser-induced pressure pulses generated by laser absorption in the cavity 12 and impinging on the vitreous material through the aperture 14. The shattered or fragmented vitreous material may be aspirated by the aspiration device, and an irrigation fluid and other media may be introduced by means of the irrigation device.

As can be seen in particular from FIG. 7, a vitrectomy needle 1 designed in accordance with the invention has, solely by performing a rotation D around the central axis Z, a comparatively large radius of action A due to its special shape, in particular due to the radial distance of the aperture 14 from the central axis Z. Due to the comparatively large radius of action A, when performing a vitrectomy, pivoting movements performed with respect to the central axis Z, through which forces may be exerted on the incision in the eye via the trocar 16, may be reduced to a minimum, combined with the advantages mentioned above.

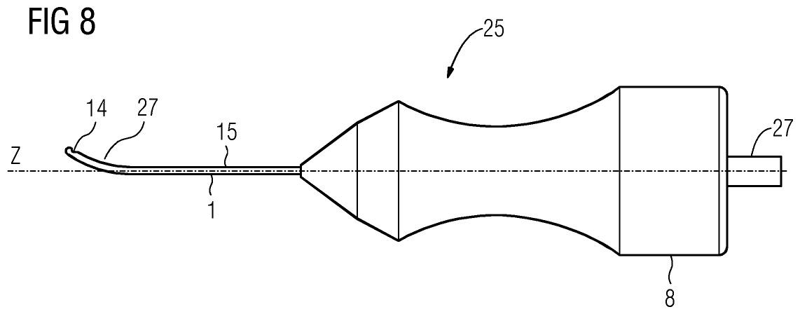

FIG. 8 shows a further embodiment of a vitrectome 25. The vitrectome 25 of FIG. 8 comprises a handle part 8 with a connection line or connection 27 for connection, for example, to a central unit 20 as schematically described in connection with FIG. 6.

The handle part 8 may be designed rotationally symmetric with respect to the central axis Z. Approximately in the middle, when viewed in longitudinal direction, the handle part 8 comprises a circumferential convex indentation or recess which is designed for holding the vitrectome 25 with two or more fingers, in particular in accordance with ergonomic aspects. In embodiments, the handle part may also have a shape other than a rotationally symmetrical shape.

In the present example, the vitrectomy needle 1 is glued to the handle part 8, and in this respect is firmly, in particular non-rotatably, connected to it.

Starting from the handle portion 8, the vitrectomy needle 1 of the embodiment of FIG. 8 has a straight section 15 which may extend, for example, over about ⅔ of the length of the vitrectomy needle 1. Adjacent to the straight section 15 is a curvature, specifically a curved section 27, which extends substantially to the distal end. In the present case, the aperture 14 is located on the inner side of the curvature, i.e. the aperture 14 is formed on the side facing the centre(s) of curvature.

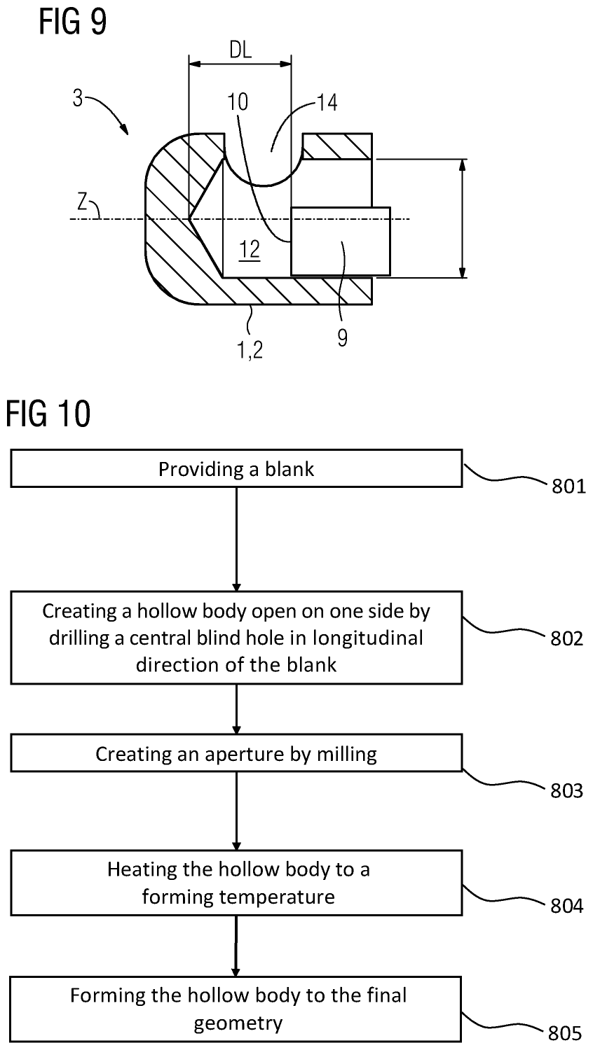

FIG. 9 shows an enlarged section of the distal end of the vitrectomy needle of the vitrectome of FIG. 8. In the present example, the axial inner surface of the distal end 3 has an approximately conical shape, which may be given, for example, by the drilling geometry of the drill used for making the hollow needle 2. The bore is essentially concentric with the central axis Z. The light-emitting surface 10 of the laser light guide 9 is spaced away from the axial inner surface of the distal end 3 by a predetermined distance DL, the cavity 12 being formed between the light-emitting surface 10 and the axial inner surface. The distance DL may be, for example, 0.9 mm to 1.2 mm, the distance DL preferably being dependent on the outer diameter and being the smaller the smaller the outer diameter.

In the present example, the light-emitting surface 14, when viewed in radial section, projects, towards the distal end 3, beyond the edge of the aperture 14 that faces away from the distal end 3. The laser light guide 9 is fixed in the hollow needle 2 or in the vitrectomy needle 1 by one or more adhesive points. The inner diameter of the hollow needle 2 may be, for example, 0.45 mm. The diameter of the laser light guide 9 is smaller than the inner diameter of the hollow needle 2, so that a channel may be formed between the laser light guide 9 and the inner wall of the hollow needle 2, for example.

In the present example, the aperture 14 is formed by a cut or incision perpendicular to the central axis Z, which may be produced, for example, by a milling tool positioned or oriented and moved perpendicular to the central axis Z.

Finally, in connection with FIG. 10, a course of a method of manufacturing a vitrectomy needle according to the invention is described.

In a first method step 801, a blank is provided which has an elongated shape and is solid. For example, a cylindrical solid blank may be used. A particularly suitable material for the blank is metal, preferably titanium or a titanium alloy. For example, titanium material referred to as Grade 4 or Grade 5 (according to ASTM standard) may be used.

In a second method step 802, a blind hole running in the longitudinal direction of the blank is drilled into the blank so that a hollow body is formed which is open on one side in the longitudinal direction, i.e. which is closed on one end.

In a third method step 803, the aperture 14, which is radial with respect to the longitudinal direction, is produced at the closed end of the blind hole by means of a milling tool in such a way that the aperture 14 opens radially into the blind hole.

In a fourth method step 804, the hollow body is heated or heated to a forming temperature, for example by resistive heating using an electric current.

In a fifth method step 805, the hollow body is formed to the desired final geometry using suitable forming tools or dies.

Finally, the laser light guide 9 may be inserted and fixed in the formed hollow body. Furthermore, a screw connection or other possibility for connection with a coupling surface for the laser light guide 9 may be attached to the proximal end 6, so that the vitrectomy needle 1 may be connected in a non-destructive, removable manner to a handle part 8, designed, for example, according to FIG. 6. Instead of a detachable connection, it may also be provided that the hollow needle 2 or the vitrectomy needle 1 is firmly connected, preferably glued, to the handle part 8.

With the method, the vitrectomy needle proposed herein may be manufactured comparatively efficiently and with high process stability and repeatability. Furthermore, by selecting appropriate forming tools, it is possible to provide different shapes and geometries for different uses and eye sizes.

In all, it becomes clear that the vitrectomy needle, the vitrectome, the vitrectomy device and the method of manufacturing a vitrectomy needle proposed herein eliminate the disadvantages in the prior art mentioned at the outset.

### LIST OF REFERENCE SIGNS

1 Vitrectomy needle
2 Hollow needle
3 Distal end
4 Vitreous body
5 Eye
6 Proximal end
7 Screw connector
8 Handle part
9 Laser light guide
10 Light-emitting surface
11 Inward-facing surface
12 Cavity
13 Wall
14 Aperture
15 Straight section
15.1 Further straight section
16 Trocar
17 Continuous curvature
18 Curvature
19 Vitrectomy device
20 Central unit
21 Control panel
22 Laser light source
23 Aspiration and irrigation device
24 Control unit
25 Vitrectome
26 Control and/or signal line
37 Curved section
801-805 Method steps
A Radius of action
Da Outer diameter
Di Inner diameter
K Radius of curvature
L Length
M Middle axis
R Minimum radial distance
Z Central axis

1-13 (canceled)

14. A vitrectomy needle comprising:
a hollow needle with a distal end for insertion into the vitreous body of an eye for performing a vitrectomy; and
a laser light guide guided in the hollow needle to the distal end with a light-emitting surface oriented towards a distal end;
wherein:
a cavity is formed in the hollow needle in the region of the light-emitting surface and the axially inward-facing surface of the distal end;
the wall of the hollow needle has an aperture in the region of the cavity which aperture extends radially with respect to the middle axis (M) of the hollow needle;
the vitrectomy needle, in particular the hollow needle, has a straight section at the proximal end;
the middle axis (M) defines a central axis (Z) in the region of the straight section; and
the middle axis (M) of the hollow needle is spaced apart from the central axis (Z) by a predetermined minimum radial distance (R) in the region of the aperture.

15. The vitrectomy needle according to claim 14, wherein, starting from the straight section towards the distal end, the hollow needle has a curvature with a, preferably constant, radius of curvature (K).

16. Vitrectomy needle according to claim 15 wherein the curvature extends from the straight section substantially to the distal end and/or wherein, starting from the straight section to the distal end, the curvature is a continuous curvature.

17. Vitrectomy needle according to claim 15, wherein, starting from the curvature towards the distal end, the hollow needle has at least one further straight section.

18. Vitrectomy needle according to claim 14, wherein the hollow needle has an outer diameter (Da) of 0.55 mm to 0.65 mm, preferably 0.6 mm, and/or wherein the hollow needle has an inner diameter (Di) of 0.35 mm to 0.55 mm, in particular from 0.35 to 0.45 mm, preferably about 0.4 mm, and/or wherein the hollow needle has a length (L), measured along the middle axis (M), of 18 mm to 30 mm, preferably 20 mm to 27 mm, further preferably about 25 mm.

19. Vitrectomy needle according to claim 14, wherein the minimum radial distance (R) is in the range of at least 5 mm to 30 mm.

20. Vitrectomy needle according to claim 14, wherein the aperture is formed on a side of the hollow needle facing the central axis (Z) or on a side of the hollow needle facing away from the central axis (Z).

21. A vitrectome comprising a handpiece and a vitrectomy needle according to claim 14, the vitrectomy needle connected to the handpiece.

22. Vitrectome according to claim 21, wherein the vitrectomy needle is detachably attached to the handpiece, in particular by means of a screw connector formed at the proximal end of the vitrectomy needle, or wherein the vitrectomy needle is firmly connected, in particular glued, to the handpiece.

23. A vitrectomy device comprising:
a laser light source;
a control unit for controlling the laser light source; and
a connection interface for connecting a vitrectome comprising a handpiece and a vitrectomy needle, the vitrectomy needle comprising a hollow needle with a distal end for insertion into the vitreous body of an eye for performing a vitrectomy, and a laser light guide guided in the hollow needle to the distal end with a light-emitting surface oriented towards a distal end;
wherein the control unit is configured for controlling at least the laser light source for performing a vitrectomy when the vitrectome is connected.

24. A method of manufacturing a vitrectomy needle with a hollow needle with a distal end for insertion into a vitreous body of an eye for performing a vitrectomy; and a laser light guide guided in the hollow needle to the distal end with a light-emitting surface oriented towards a distal end, the method comprising:
providing an elongated solid, in particular cylindrical, blank;
creating a hollow body from the blank by drilling a central blind hole extending in longitudinal direction of the blank by means of a drilling tool;
creating the aperture, which is radial with respect to the longitudinal direction, at the closed end of the blind hole by means of a milling tool, in such a way that the aperture opens radially into the blind hole;
heating the hollow body to a forming temperature, in particular in the range between 60° C. and 140° C., in particular between 100° C. and 130° C., further in particular at about 120° C.; and
forming of the hollow body to the final geometry of the vitrectomy needle by means of a forming tool.

25. The method according to claim 24, wherein the blank has an outer diameter of 0.2 mm to 1 mm.

26. The method according to claim 24, wherein the heating comprises resistive electrical heating by means of electric current and/or inductive heating.
